(19) United States
(12) Patent Application Publication
(10) Pub. No.: US 2012/0284815 A1
(43) Pub. Date: Nov. 8, 2012

(54) APOMICTIC SOYBEAN PLANTS AND METHODS FOR PRODUCING

(76) Inventor: GEORGE ROBERT TAYLOR, WEST LAFAYETTE, IN (US)

(21) Appl. No.: 13/463,632

(22) Filed: May 3, 2012

Related U.S. Application Data
(63) Continuation-in-part of application No. 13/102,591, filed on May 6, 2011.

(51) Int. Cl. A01H 5/00; A01H 5/10; A23L 1/20; C07K 1/14; C11B 1/00; C12N 5/04; A01H 1/02; A01H 1/06

(52) U.S. Cl. 800/260; 800/312; 800/278; 800/279; 800/281; 800/284; 800/300; 800/303; 800/302; 800/301; 530/378; 554/9; 435/415; 426/443

(57) ABSTRACT

The present invention provides apomictic soybean varieties and methods of making the same. The present invention relates to apomictic soybean plants having a mutant allele designated AP1, which confers apomixis. The invention relates to crossing soybean plants containing the AP1 allele to produce novel types and varieties of apomictic soybean plants.

This application is a continuation-in-part application of prior U.S. application Ser. No. 13/102,591, filed May 6, 2011 hereby incorporated by reference in its entirety.

### BACKGROUND OF THE INVENTION

The present invention generally provides apomictic soybean plants and more particularly, apomictic soybean plants having an average percent conversion greater than 51% for conversion of flowers to pods or greater than 60% for conversion of flowers to pods and an average of at least 51 pods per plant or at least 60 pods per plant or at least 100 pods per plant. The present invention further provides methods for producing apomictic soybean plants. All publications cited in this application are herein incorporated by reference.

There are numerous steps in the development of any novel, desirable plant germplasm. Plant breeding begins with the analysis and definition of problems and weaknesses of the current germplasm, the establishment of desired goals, and the definition of specific breeding objectives. The next step is selection of germplasm that possess the traits to meet the desired goals. The goal is to combine in a single variety an improved combination of desirable traits from the parental germplasm. These important traits may include higher seed yield, resistance to diseases and insects, better stems and roots, tolerance to drought and heat, better agronomic quality, resistance to herbicides, and improvements in compositional traits.

Reproduction in plants is ordinarily classified as sexual or asexual. The term apomixis is generally accepted as the replacement of sexual reproduction by various forms of asexual reproduction (Rieger et al, In Glossary of Genetics and Cytogenetics, Springer-Verlag, New York, N.Y., 1976). Apomixis is a genetically controlled method of reproduction in plants where the embryo is formed without union of an egg and sperm nuclei. In most forms of apomixis, pseudogamy or fertilization of the polar nuclei to produce endosperm is necessary for seed viability. Obligate apomicts are believed to have a completely closed recombination system; that is, recombination occurs only during microsporogenesis and is absent during megasporogenesis. In facultative apomicts, both apomictic and sexual modes of reproduction coexist. All known mechanisms of apomixis share three developmental components: the generation of a cell capable of forming an embryo without prior meiosis (apomeiosis); the spontaneous, fertilization-independent development of the embryo (parthenogenesis); and the capacity to either produce endosperm autonomously or to use an endosperm derived from fertilization (Koltunow, A. M., Plant Cell 5, 1425-1437, 1993 and Carman J. G., Biol. J. Linn. Soc. 61, 51-94, 1997).

The use of apomixis in plant breeding has economic potential because it can cause any genotype, regardless of how heterozygous, to breed true. Apomixis is a reproductive process that bypasses female meiosis and syngamy to produce embryos genetically identical to the maternal parent.

Soybean, Glycine max (L), is a valuable field crop. Thus, a continuing goal of plant breeders is to develop stable, high yielding soybean varieties that are agronomically sound. The reasons for this goal are to maximize the amount of grain produced on the land used and to supply food for both animals and humans. To accomplish this goal, the soybean breeder must select and develop soybean plants that have the traits that result in superior varieties. Current known varieties of soybeans show an average percent conversion of less than 50% for conversion of flowers to pods and no more than an average of 50 pods per plant.

Therefore it would be desirable to have a variety of soybeans that have a higher percent conversion of flowers to pods and a greater number of pods per plant. It would further be desirable if there were a facile method for producing such a variety.

### SUMMARY OF THE INVENTION

In one aspect of the present invention there is provided a method of producing a soybean apomictic plant that exhibits a greater percent conversion of flowers to pods and a greater number of pods per plant than commercial soybean varieties comprising the steps of selecting a plurality of double ovule seeds; growing said seeds into soybean plants; and selecting plants having a percent conversion greater than about 51% and at least 51 pods per plant or selecting plants having a percent conversion greater than about 60% and at least 60 pods per plants or selecting plants having a percent conversion greater than about 60% and at least 100 pods per plant. The selected plants have the additional characteristic of being apomictic.

In another aspect of the present invention there is provided further steps to the method of producing a soybean apomictic plant that exhibits an increased hybrid vigor compared to the parent plant comprising cross-breeding the selected apomictic soybean plant as a male with a different soybean plant as the female; selecting a plurality of seeds from the female soybean plant; growing said seeds into soybean plants; and selecting plants having a hybrid vigor of greater than about 30% and at least 60 pods per plant or selecting plants having a hybrid vigor of greater than about 50% and at least 60 pods per plant or selecting plants having a hybrid vigor of greater than about 50% and at least 100 pods per plant. The selected plants have the additional characteristic of being apomictic.

In a further aspect of the present invention there is provided apomictic soybean plants of variety S-201, CN-0629 and 7B1. Also provided are the seeds and progeny of these varieties.

In yet another aspect of the present invention there is provided a method of producing soybean seed, comprising crossing a plant of soybean variety S-201 with itself or a second soybean plant, representative seed of said soybean variety having been deposited at the American Type Culture Collection (ATCC) and assigned Accession No. PTA-11892.

In a further aspect of the present invention there is provided a method of producing a plant of soybean variety S-201 comprising an added desired trait, the method comprising introducing a transgene conferring the desired trait into a plant of soybean variety S-201, representative seed of said soybean variety having ATCC Accession No. PTA-11892.

In another aspect of the present invention there is provided method of producing an inbred soybean plant derived from the soybean variety S-201, the method comprising the steps of preparing a progeny plant derived from soybean variety S-201 by crossing a plant of the soybean variety S-201 with a soybean plant of a second variety, representative seed of said soybean variety S-201 having ATCC Accession No. PTA-11892; crossing the progeny plant with itself or a second plant to produce a seed of a progeny plant of a subsequent generation; growing a progeny plant of a subsequent generation from said seed and crossing the progeny plant of a subsequent generation with itself or a second plant; and repeating the last two steps until an inbred soybean plant derived from the soybean variety S-201 is produced.

These and other features, aspects and advantages of the present invention will become better understood with reference to the following drawings, description and claims.

### BRIEF DESCRIPTION OF THE DRAWINGS

FIG. 1 is a photograph showing an example of selected double ovule seeds from soybean variety S-201;

FIG. 2 is a photograph showing an example of a pair of end-to-end double ovule seeds from soybean variety CN-0629;

FIG. 3 is a photograph showing an example of end-to-end and side-to-side double ovules from soybean variety 7B1;

FIG. 4 is a photograph showing the uniformity of several soybean plants of soybean variety S-201 grown for selection;

FIG. 5 is a photograph showing the typical percent conversion for an apomictic soybean variety according to the present invention;

FIG. 6 is a photograph showing two 7B1 plants with more than 150 pods per plant;

FIG. 7 is a photograph showing a single node of a 7B1 plant having 16 pods;

FIG. 8 is a photograph showing a single node of a 7B1 plant having a high percent conversion of flowers to pods;

FIG. 9 is a photograph of the 7B1 plant of the line shown in FIGS. 6,7 and 8;

FIG. 10 is a photograph showing 90 pods on 9 nodes of a 7B1 plant; and

FIG. 11 is a photograph showing 38 pods on 3 nodes of a 7B1 plant.

### DETAILED DESCRIPTION OF THE INVENTION

In the description and examples that follow, a number of terms are used. In order to provide a clear and consistent understanding of the specification and claims, including the scope to be given such terms, the following definitions are provided. If no definition is provided, all other technical and scientific terms used herein have the same meaning as is commonly understood by one of skill in the art to which the invention belongs.

Allele. An allele is any of one or more alternative forms of a gene which relate to one trait or characteristic. In a diploid cell or organism, the two alleles of a given gene occupy corresponding loci on a pair of homologous chromosomes.

Apomictic. As used herein, “apomictic” describes a plant that reproduces using apomixis

Apomixis. Asexual reproduction in organisms that are also able to reproduce sexually, in which embryos are formed without fertilization or the creation of specialized reproductive cells.

Backcrossing. Backcrossing is a process in which a breeder repeatedly crosses hybrid progeny back to one of the parents, for example, a first generation hybrid F₁ with one of the parental genotypes of the F₁ hybrid.

Crossbreeding. As used herein, “crossbreeding” refers to the act of mating (crossing) individuals of different species or varieties of plants to produce hybrids.

Determinate growth. Growth in which the axis, or central stem, being limited by the development of the floral reproductive structure, does not grow or lengthen indefinitely during the growing season. Plants with determinate growth have shorter flowering periods and produce pods along the entire length of the stem.

Double ovule seed. Production of two viable seeds sharing one hilum in a pod. One seed is termed a normal seed and the second seed is termed a “sister cell”. Typically, only an extremely small percent of soybean plants produce double ovule seeds.

Ectopic expression. Expression of a gene out of its expected time or place.

Emasculation. The removal of male flowers or anthers to prevent self-pollination.

Embryo. The embryo is the small plant contained within a mature seed.

Gene. As used herein, “gene” refers to a segment of nucleic acid. A gene can be introduced into a genome of a species, whether from a different species or from the same species, using transformation or various breeding methods.

Genotype. As used herein, “genotype” refers to the genetic constitution of a cell or organism.

Growing season. The time from planting seed in the spring until harvesting the crop in the fall.

Hilum. This term refers to the scar left on the seed that marks the place where the seed was attached to the pod prior to the seed being harvested.

Hybrid. Heterozygous offspring of two parents that differ in one or more inheritable characteristics.

Hybrid vigor. This term refers to the high or improved performance (usually in terms of yield) expressed by interspecific hybrids, resulting from their genetic heterogeneity. This high performance is typically lost in hybrid offspring, forcing farmers to obtain new hybrid seed for every planting.

Hypocotyl. A hypocotyl is the portion of an embryo or seedling between the cotyledons and the root. Therefore, it can be considered a transition zone between shoot and root.

Indeterminate growth. Growth in which the axis or central stem continues to grow or lengthen during the growing season and flowering usually lasts from 30-38 days as the stem continues to grow. Plants with indeterminate growth have long flowering periods and produce no pods on the last three nodes at the terminal end of the stem.

Main stem. The part of the plant from the roots to the terminal end that has nodes, in which some bear the branches and some just flowers and pods. It is the only part of the plant that is stem.

Maturity Date. Plants are considered mature when 95% of the pods have reached their mature color. The number of days is calculated either from August 31 or from the planting date.

Maturity Group. This term refers to an agreed-on industry division of groups of varieties based on geographic zones in which they are adapted, primarily according to day length or latitude. They consist of very long day length varieties (Groups 000, 00, 0), and extend to very short day length varieties (Groups VII, VIII, IX, X).

Megaspore mother cell. The precursor to the ovule; the single cell that, following fertilization, divides into 2 equal cells, that divide into 4 cells, that divide into 8 cells, etc. until they differentiate into a seed. In apomixis, the megaspore mother cell is stimulated to begin division without fertilization and will produce 2 cells, 4 cells, 8 cells, etc., which all contain the same exact genetic material that is identical to that in the megaspore mother cell. Differentiation occurs as with normal cells and viable seed is produced.

Meiosis. Cell division that produces reproductive cells in sexually reproducing organisms; the nucleus divides into four nuclei each containing half the chromosome number.

Node. The point on a plant stem from which the leaves, pods or lateral branches grow.

Ovule. A single cell that contains all of the genetic material of the female plant.

Percent conversion. As used herein, the term “percent conversion” refers to the average percentage of flowers on a soybean plant that is converted or develop into pods.

Plant. As used herein, the term “plant” includes reference to an immature or mature whole plant, including a plant from which seed, grain, or anthers have been removed. Seed or embryo that will produce the plant is also considered to be the plant.

Plant Height. Plant height is taken from the top of the soil to the top point or axis of the plant and is measured in inches.

Plant Parts. As used herein, the term “plant parts” (or a soybean plant, or a part thereof) includes but is not limited to protoplasts, leaves, stems, roots, root tips, anthers, pistils, seed, grain, embryo, pollen, ovules, cotyledon, hypocotyl, pod, flower, shoot, tissue, petiole, cells, meristematic cells, and the like.

Pod. This term refers to the fruit of a soybean plant. It consists of the hull or shell (pericarp) and the soybean seeds inside the hull.

Polyploid. A plant that receives more than one set of chromosomes from each of its parents.

Progeny. As used herein, includes an F₁ soybean plant produced from the cross of two soybean plants and progeny further includes, but is not limited to, subsequent F₂, F₃, F₄, F₅, F₆, F_{7,} F_{8,} F_{9,} and F₁₀ generational crosses with the recurrent parental line.

Raceme. An inflorescence in which the flowers are borne along the main stem, with the oldest flowers at the base. The raceme can be simple or compound.

Relative Maturity (RM). The term relative maturity is a numerical value that is assigned to a soybean variety based on comparisons with the maturity values of other soybean varieties. The number preceding the decimal point in the RM refers to the maturity group. The number following the decimal point refers to the relative earliness or lateness within each maturity group. For example, a 3.0 is an early group III variety, while a 3.9 is a late group III variety.

Seed Yield (Bushels/Acre). The yield in bushels/acre is the actual yield of the grain at harvest.

Seeds Per Pound. Soybean seeds vary in seed size; therefore, the number of seeds required to make up one pound also varies. The number of seeds per pound affects the pounds of seed required to plant a given area and can also impact end uses.

Sister cell. A sister cell is a twin of the normal megaspore mother cell that occurs at the exact same time in the development process. Usually the genetic make-up of the sister cell matches exactly the genetic make-up of the megaspore mother cell. If both get fertilized, twin seeds that are identical are produced. If one is not fertilized, which is the normal procedure, it withers and dies; however, very rarely the sister cell may continue production of seed without fertilization, resulting in a double ovule seed. Prior to the mutant allele of the present invention, a double ovule seed occurs in approximately 1 seed in 5 billion.

Syngamy. The fusion of two gametes in fertilization.

Transgene. A gene that is transferred from an organism of one species to an organism of another species by genetic engineering.

The following detailed description is of the currently contemplated modes of carrying out the invention. The description is not to be taken in a limiting sense, but is made merely for the purpose of illustrating the general principles of the invention, since the scope of the invention is best defined by the appended claims.

Broadly, the present invention is directed toward apomictic soybean varieties, including both plants and seeds. In one embodiment, the apomictic soybean plants of the present invention have a percent conversion of flowers to pods of greater than about 51% and at least 51 pods per plant or at least 60 pods per plant under normal growing conditions. In another embodiment, the apomictic soybean plants of the present invention have a percent conversion of flowers to pods of greater than about 60% and at least 60 pods per plant under normal growing conditions. It will be appreciated by those skilled in the art that these values may vary due to environmental parameters beyond the control of the skilled artisan.

The present invention is also directed to a method for producing the apomictic soybean varieties of the present invention where characteristics of any variety can be stabilized through the use of apomixis. In one embodiment, the seeds are grown and the resulting plants are selected for the desired traits including higher yield through a higher percent conversion of flowers to pods of greater than about 51% and at least 51 pods per plant or at least 60 pods per plant. In one embodiment, the seeds are grown and the resulting plants are selected for the desired traits including higher yield through a higher percent conversion of flowers to pods of greater than about 60% and at least 60 pods per plant.

A hybrid soybean variety can be the cross of two inbred lines, each of which may have one or more desirable characteristics lacked by the other or which complement the other. The hybrid progeny of the first generation is designated F₁. In the development of hybrids only the F₁ hybrid plants are sought. The hybrid is more vigorous than its inbred parents. This hybrid vigor, or heterosis, can be manifested in many ways, including increased vegetative growth and increased yield.

With apomictic reproduction, progeny of specially adaptive or hybrid genotypes would maintain their genetic fidelity throughout repeated life cycles. In addition to fixing hybrid vigor, apomixis can make possible commercial hybrid production in crops where efficient male sterility or fertility restoration systems for producing hybrids are not known or developed. In addition, apomixis can increase the reproductive capacity of plants with distorted or adverse chromosome constitutions, especially where humans have changed the gene content of said chromosomes.

Apomixis can make hybrid development more efficient by eliminating the need for multiple crosses. It also simplifies hybrid production and increases genetic diversity in plant species with good male sterility systems. It would be ideal to find genes controlling obligate or a high level of apomixis in the cultivated species and be able to readily hybridize cross-compatible sexual x apomictic genotypes to produce true-breeding F₁ hybrids.

Although apomixis is effectively used in Citrus to produce uniform and disease- and virus-free rootstock (Parlevliet J. E. et al., in Citrus. Proc. Am. Soc. Hort. Sci., Vol. 74, 252-260, 1959) and in buffelgrass (Bashaw, Crop Science, Vol. 20, 112, 1980) and Poa (Pepin et al., Crop Science, Vol. 11, 445-448, 1971) to produce improved cultivars, it has not been successfully transferred to a cultivated crop plant. The transfer of apomixis to important crops would make possible development of true-breeding hybrids and commercial production of hybrids without a need for cytoplasmic-nuclear male sterility and high cost, labor-intensive production processes. An obligate apomictic F₁ hybrid would breed true through the seed indefinitely and could be considered a vegetative or clonal method of reproduction through the seed. The development of apomictically reproducing cultivated crops would also provide a major contribution toward the food security in developing nations (Wilson et al., In Proceedings of the International Workshop on Apomixis in Rice, Changsha, People's Republic of China, 13 Jan.-15 Jan., 1992. Hunan Hybrid Rice Research Center, Changsha, People's Republic of China).

Commercial soybean plants and seeds currently being grown have an average percent conversion of no greater than 50% for conversion of flowers to pods. A current commercial soybean plant has up to 100 flowers and will have no more than about 50 pods per plant at harvest time. This limits the amount of soybeans currently produced per acre. In one embodiment, the apomictic soybean plants of the present invention have an average of at least 60 pods per plant and often more than 300 or 400 pods per plant. In another embodiment, the apomictic soybean plants of the present invention have an average of at least 100 pods per plant and often more than 300 pods per plant. Moreover, production of the soybean varieties of the present invention are produced in a shorter time frame than normal breeding used to produce new varieties because of the apomixes in one parent of the hybrid cross. The apomictic soybean varieties of the present invention meet the desire to increase the yield of soybeans per acre from current levels through the use of hybrid vigor.

In one embodiment of the present invention there is provided a method for producing apomictic soybean varieties having a higher percent conversion of flowers to pods and a greater number of pods overall as compared to current varieties of soybean. The method comprises the steps of selecting double ovule seeds from seed lots of sources with desired traits of soybean germplasm. The number of seeds required can vary depending upon the viability of both ovules of the seed. Double ovule seeds result from the development of sister cells along the side or end of a normal single ovule at any location in the pod and must share the same hilum. Double ovule seeds occur at a comparatively high frequency but typically go unobserved because of a high death rate for the sister cells. The occurrence of a surviving sister cell is estimated to be one in 5 billion or more for soybeans.

To find just one double ovule seed in normal soybeans usually takes more than one year. Even if a dozen or so double ovule seeds are found in the same year, they will be exactly like the parent line and have no value at all. However, once in many years a double ovule seed can be produced that has value because it produces seed without a contribution from a male soybean plant; that is, it needs no pollen to produce seed. Instead, the megaspore mother cell initiates division of an ovule on its own; whereas normally division does not begin until after fertilization. When the production of a new plant is complete, all the contribution of genes comes from the female plant and will breed true—all traits will be the same as those of the female plant, as the traits were characteristics of the sister cell's genes and do not change. This property is apomixis.

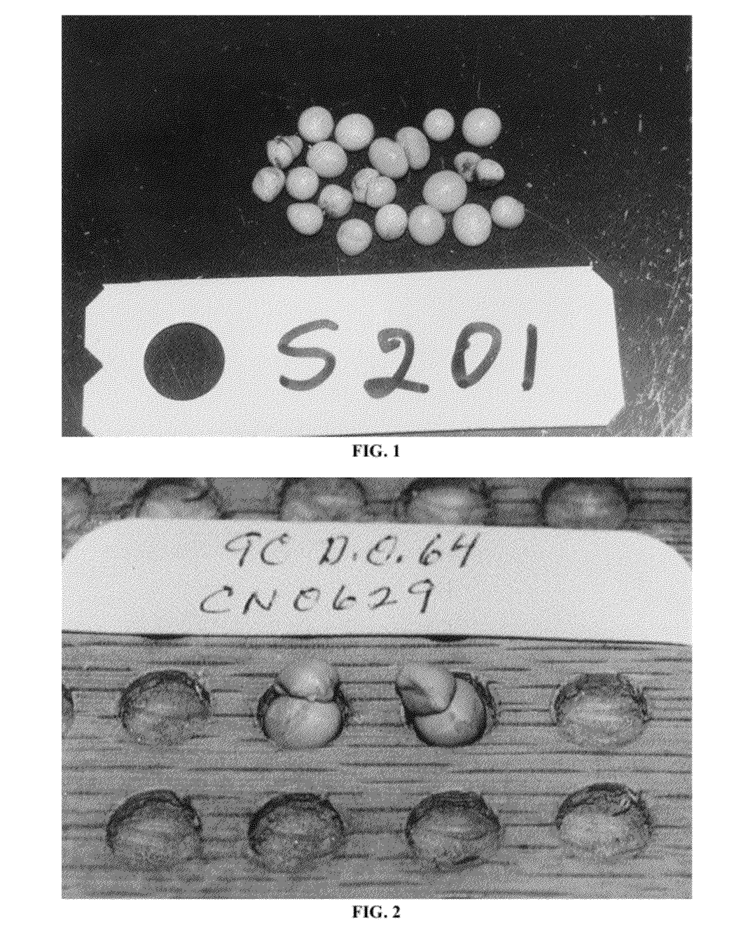

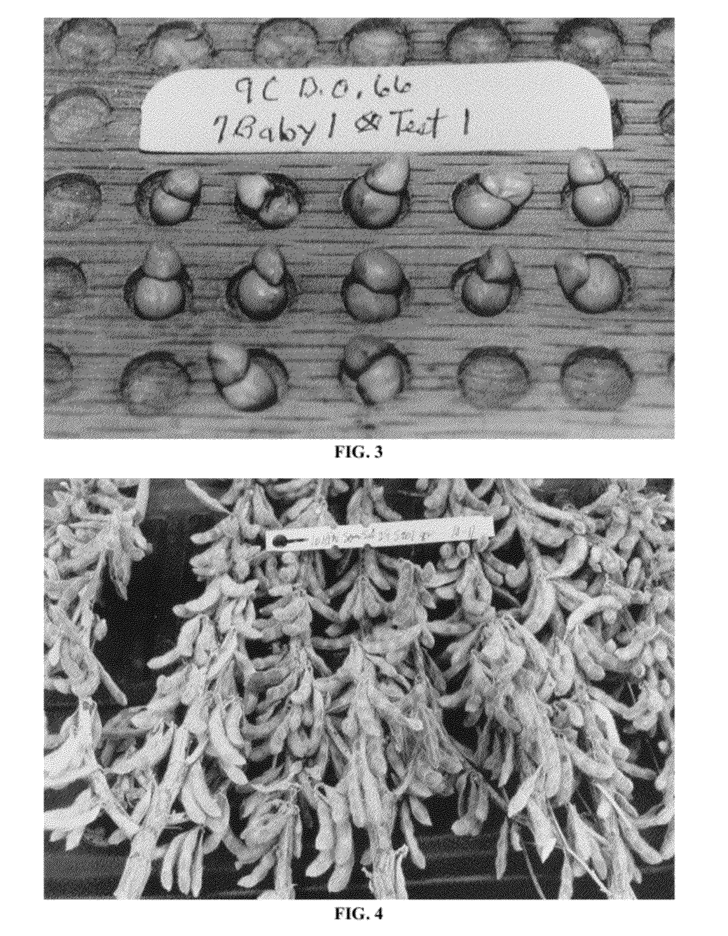

In an illustrative embodiment the number of double ovule seeds required for economic development purposes may be from about 10,000 to 30,000. However, it will be appreciated that the method of the present invention is not limited to the number of double ovule seeds initially selected. Examples of double ovule seeds are shown in FIGS. 1-3 for three different varieties of apomictic soybeans of the present invention. The double ovule seeds may be end to end or side to side. It has not been observed to make a difference as to the orientation of the double ovules. Through a soybean variety development program comprising conventional cross breeding of soybean varieties, selection, selfing and/or further cross breeding and further selection, a line designated S-201 was developed. It was discovered that the S-201 variety had a high frequency of double ovule seeds compared to conventional soybean varieties. Following the harvest of the 2009 crop, a concerted effort was made to find as many double ovule pairs as possible. Sorting through several bushels of seed of S-201 and its derivatives produced more than 3,200 different double ovule seed pairs. These formed the basis of most of the recent studies concerning the inheritance of double ovule seeds and apomixis.

In the next step, the seeds are grown out and observed for the percent conversion of flowers to pods and the total number of pods per plant. Lines with higher-than-normal numbers for these traits are selected to be used to produce the apomictic plants of the present invention. For example, in one embodiment, plants may be selected having a percent conversion of greater than about 51%. In another embodiment, plants may be selected having a percent conversion of greater than about 60%. In an additional embodiment, plants may be selected having a percent conversion of greater than about 70%. In an illustrative embodiment, plants may be selected having a percent conversion greater than about 80% or even about 90% or even approaching about 100%. In some embodiment, plants may be selected having a percent conversion of between about 51% and about 70%. In other embodiments, plants may be selected having a percent conversion of between about 60% and about 70%. In additional embodiments, plants may be selected having a percent conversion of between about 70% and about 80% or between about 70% and 90%. In further embodiments, plants may be selected having a percent conversion of between about 90% and about 100%. Additionally, the number of pods per plant may be greater than about 60 pods, or greater than aboutl00 pods, or greater than about 200 pods, or greater than about 300 pods, or even greater than about 400 pods. As shown herein, plants have been produced by the method of the present invention having greater than 400 pods per plant. In some embodiments, the plants may have between about 60 and about 100 pods per plant. In other embodiments, the plants may have between about 100 and about 200 pods per plant. In additional embodiments, the plants may have between about 200 and about 300 pods per plant. In further embodiments, the plants may have between about 300 and about 400 pods per plant. In other embodiments, the plants may have greater than about 400 pods per plant. The plants may also be tested for apomixis, which involves emasculation of all the anthers of several plants of the line being tested and the observation of pods set by the emasculated flowers. It has been discovered that most, if not all, of the plants derived from the double ovule seeds of S-201, as well as, the progeny plants derived from the S-201 variety also have the characteristic of being apomictic.

FIG. 4 shows an example of one of the varieties produced by this method. S-201 unexpectedly had a percent conversion of flowers to pods approaching 100%, and on average of 92%. S-201 also unexpectedly produced a higher than normal amount of double ovule seeds. In one embodiment, S-201 produced a higher than normal amount of double ovule seeds having between about 1 and about 2 double ovule seeds per thousand seeds overall. In another embodiment, S-201 produced a higher than normal amount of double ovule seeds having between about 1 and about 3 double ovule seeds per thousand seeds overall. In an additional embodiment, S-201 produced a higher than normal amount of double ovule seeds having between about 1 and about 4 double ovule seeds per thousand seeds overall. In a further embodiment, S-201 produced a higher than normal amount of double ovule seeds having between about 1 and about 5 double ovule seeds per thousand seeds overall. On average, S-201 produced about 1.3 double ovule seeds per thousand seeds overall. Current soybean varieties have frequencies of double ovules of approximately 1 seed in 5 billion.

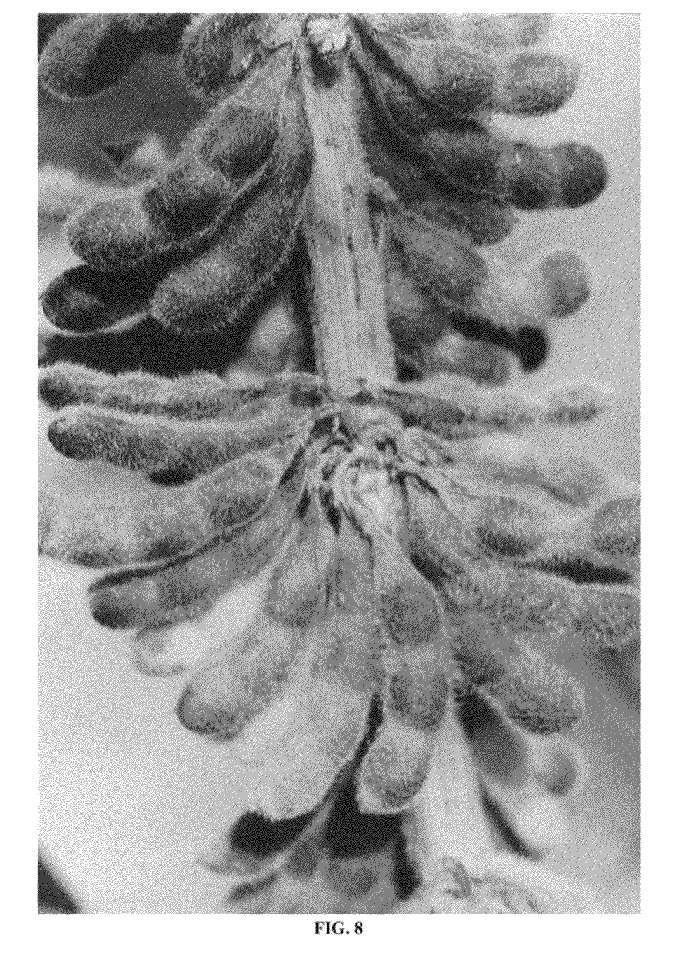

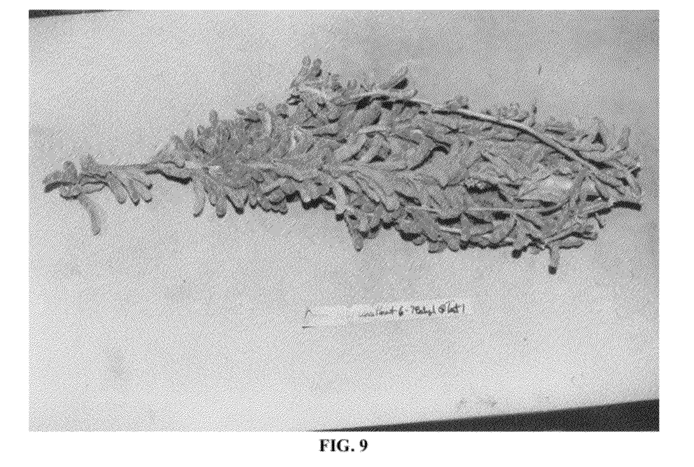

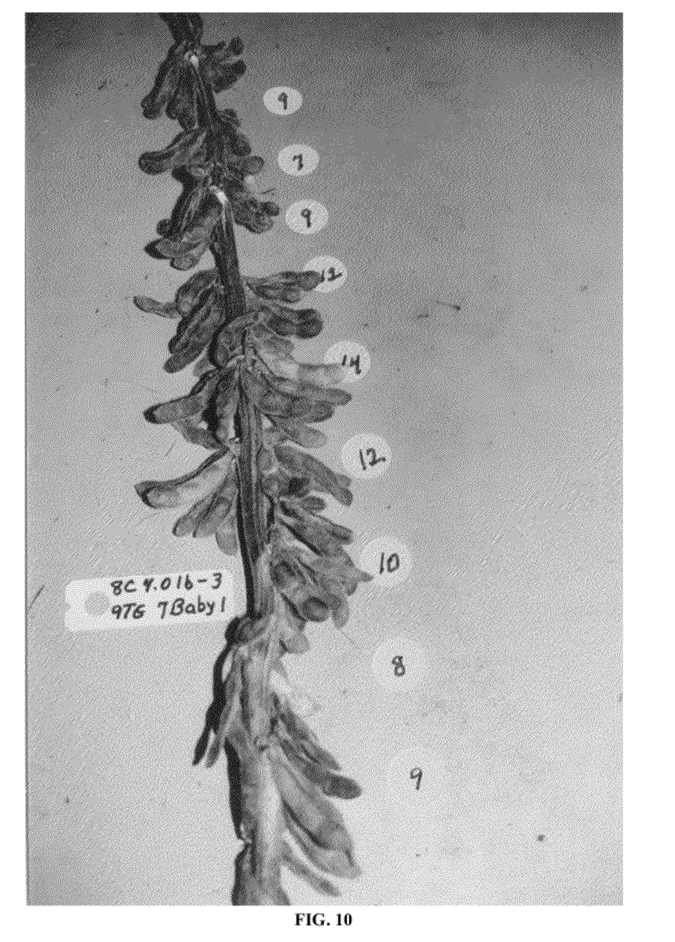

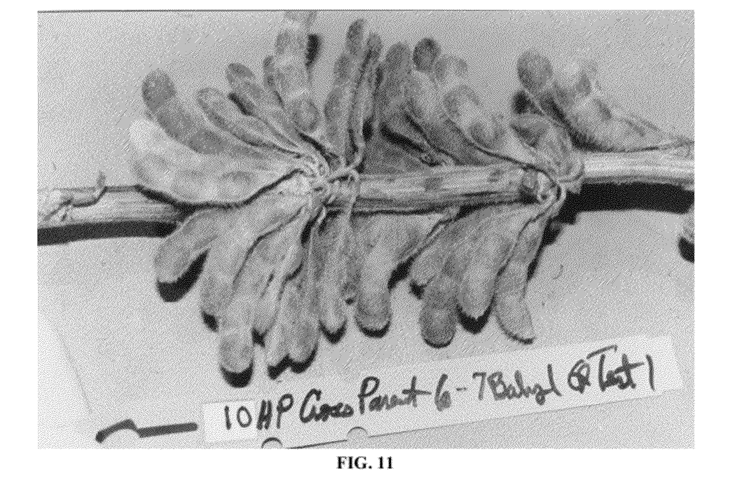

Double-ovule seeds from selected plants were grown the following year and emasculation was performed on 10 plants selected at random, indicated as variety 7B1 (FIGS. 6-11). All the pollen was removed from 10 flowers on each of the plants and tags indicated where each emasculated flower was located. Apomixis occurs when an ovule begins dividing producing seed without the pollination that is normally required. It was concluded that the seed produced after the flowers were emasculated was due to apomixis. No pollen should have been available to the flowers on the selected plants because the anthers were removed long before the pollen was mature. The plants grown from the first pods produced on those plants have continued as apomictic single varieties for at least three years with significant uniformity observed in the plants each year overall from seed produced in 2009. In addition, three of the ten original plants have produced progeny that had at least one double ovule.

Without being bound by any theory, it is believed that this high production of double ovule seeds in S-201 is due to the presence of a mutant allele which arose during the development of the S-201 variety. This mutant allele may act alone or in concert with other genes present in S-201 to provide a higher frequency of double ovule seeds. It is further believed that this mutant allele may also have an effect on the apomictic characteristic of the plants and progeny plants of the present invention.

In another embodiment of the present invention, the method may further comprise the steps of crossbreeding selected plants of soybean variety S-201 or its progeny with another soybean variety with desired traits. In an illustrative example, the plants of soybean variety S-201 or its progeny are used as male plants and the additional varieties are used as female plants. The seeds from the female plants are then harvested and grown. Plants are then selected for the desired traits as described above. This step may be repeated as many times as desired to obtain soybean plants with the added desired traits. The added desired traits may be, but not limited to, male sterility, herbicide tolerance, insect or pest resistance, disease resistance, modified fatty acid metabolism, modified carbohydrate metabolism and modified soybean fiber characteristics. In an alternate example the added trait may be herbicide tolerance where the tolerance is conferred to an herbicide such as, but not limited to, glyphosate, sulfonylurea, imidazalinone, dicamba, glufosinate, phosphinothricin, phenoxy proprionic acid, cycloshexone, triazine, benzonitrile and broxynil. (See U.S. Pat. Nos. 7,868,231, 7,868,232 and 7,572,958, all herein incorporated by reference in the entirety).

In another embodiment, the plants of soybean variety S-201 or its progeny are used as female parents, and additional varieties are used as male parents.

In an alternate environment, desired traits may be conferred to the plants of soybean variety S-201 and/or its progeny by other methods such as recombinant techniques. Such methods are well known in the art.

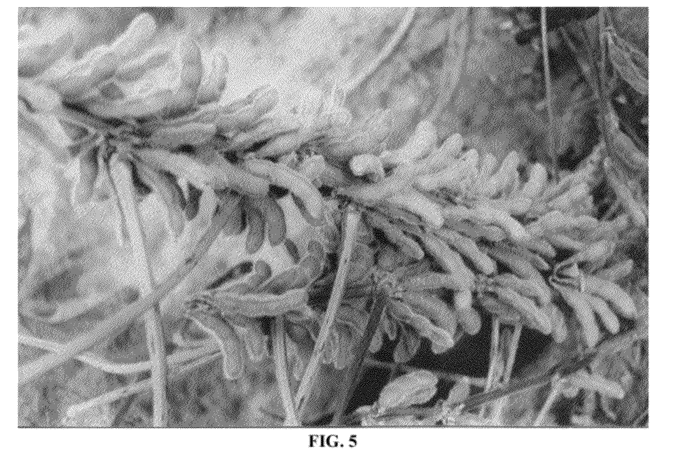

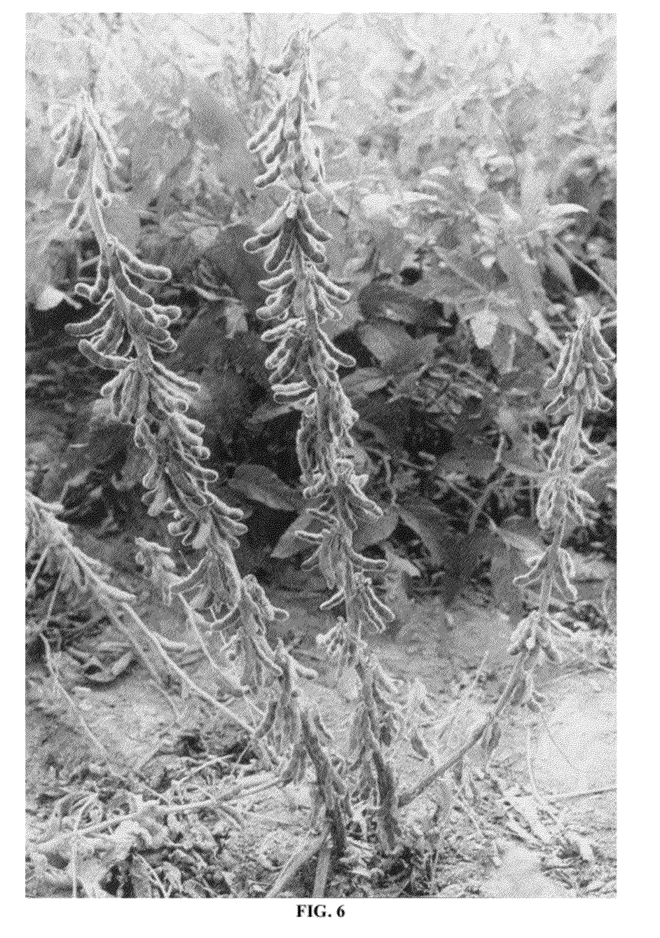

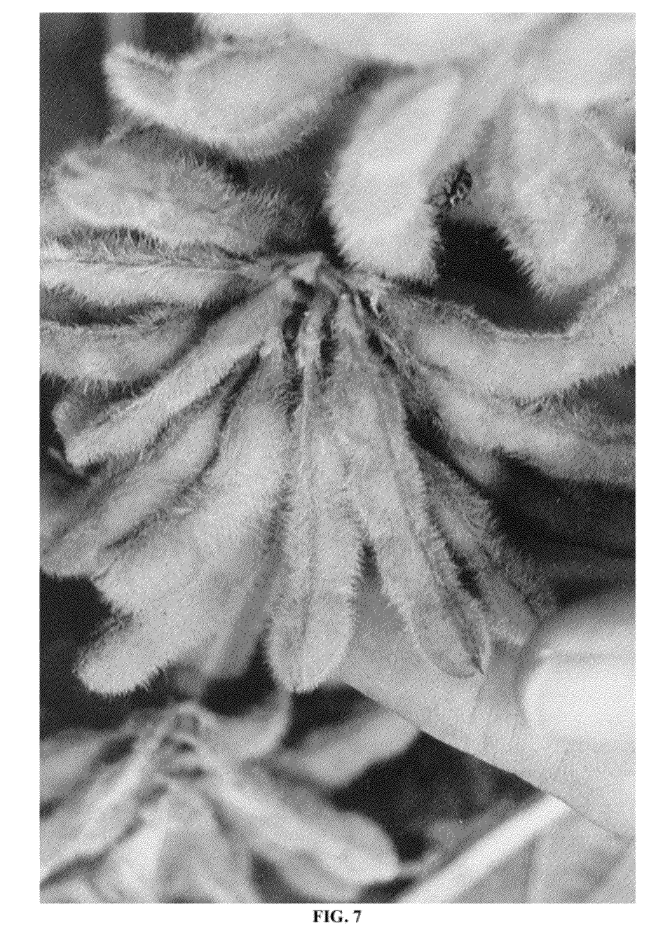

Another apomictic variety of soybean was selected from the F₄ progeny of S-201 and labeled CN-0629. This experimental line CN-0629 was subsequently used to derive new lines, many which have produced more than 400 pods per plant. In crossbreeding of CN-0629, S-201, or 7B1, 61 different lines were used as females in crosses involving selected progeny from CN-0629, S-201, or 7B1 as males. The F₁ generation from this crossbreeding showed considerable variation in percent conversion and the number of pods. Most of the F₁ generation showed a phenotypic similarity when grown a year later. One such progeny was the variety 7B 1 shown in FIGS. 5-11. The Figures show the increased number of pods per plant, particularly at each individual node.

Another characteristic of the apomictic soybean variety S-201 and its progeny is that they have determinate growth characteristics. Nearly all soybean varieties exhibit one of two possible growth habits: determinate or indeterminate growth. Most indeterminate varieties are adapted to maturity group IV and earlier. These varieties are usually grown in the northern part of the United States. These varieties have overlapping vegetative and reproductive growth periods. Usually less than one-half of the main stem nodes have developed when flowering begins. Stem growth continues as flowering progresses up the stem. Indeterminate stems do not end with a terminal raceme or the raceme is much reduced in size. Flowers and pods develop at different times and rates depending on node locations. Nodes with the earliest flowers and slowest developing pods are located near the bottom of the stem.

Most varieties with a determinate growth habit are classified in maturity groups V through X and are normally grown in the southern part of the United States and do not grow well further north. These varieties have rather distinct vegetative and reproductive development periods. Few stem nodes develop once flowering begins and the stem ends with a terminal raceme. Flowers and pods tend to develop at about the same time and rate for all stem nodes. A few varieties with determinate growth habit have been released for northern soybean production areas. These varieties are usually classified as semi-dwarf and obtain about one-half to two-thirds the height of other adapted varieties.

A unique characteristic of the apomictic soybean varieties of the present invention is that they have determinate growth habits and have stems with a terminal raceme. However, unlike the usual soybeans with determinate growth habits, the soybean varieties of the present invention show a hardiness that allows them to grow in the northern part of the United States. Moreover, in contrast to both usual varieties with either determinate or indeterminate growth characteristics, the soybean varieties of the present invention have a higher percent conversion of flowers to pods and a greater number of pods per plant.

The apomictic plant of the current invention in part results from the mutant allele of the present invention, termed AP1 herein, which will advantageously be introduced into varieties that contain desirable genetic traits such as resistance to disease, drought tolerance, heat and/or cold tolerance, and the like to produce new apomictic plants containing the desirable trait that will breed true.

The genetic factor of the present invention is capable of transmitting the characteristic of a higher than normal production of double ovule seeds which are useful in producing the apomictic plants of the present invention, as well as a higher than normal percent conversion of flowers to pods and higher than normal number of pods. The AP1 mutant appears to be a single dominant or partially dominant mutant allele. Alternatively, the mutant allele may be governed by a dominant gene with additional modifier genes influencing the level of expression. Observations made of the F₁ and F₂ progeny of crosses involving S-201 and 7B1 indicate a small number of genes is likely responsible for the “partial dominance” observed, or it may be a dominant gene with several modifiers of different strengths. Such a relationship between genes may have appeared with the frequency reported because of a mutation in one or more alleles. Such a mutation may have permitted the expression of a previously unobserved phenomenon. It is a feature of the present invention that this mutant allele may be used in and transferred to other soybean varieties.

It should be understood, of course, that the foregoing relates to exemplary embodiments of the invention and that modifications may be made without departing from the spirit and scope of the invention as set forth in the following claims.

### FURTHER EMBODIMENTS OF THE INVENTION

The advent of new molecular biological techniques has allowed the isolation and characterization of genetic elements with specific functions, such as encoding specific protein products. Scientists in the field of plant biology developed a strong interest in engineering the genome of plants to contain and express foreign genetic elements, or additional, or modified versions of native or endogenous genetic elements in order to alter the traits of a plant in a specific manner. Any DNA sequences, whether from a different species or from the same species, which are introduced into the genome using transformation or various breeding methods are referred to herein collectively as “transgenes.” In some embodiments of the invention, a transgenic variant of apomictic soybean may contain at least one transgene but could contain at least 1, 2, 3, 4, 5, 6, 7, 8, 9, 10, and/or no more than 15, 14, 13, 12, 11, 10, 9, 8, 7, 6, 5, 4, 3, or 2. Over the last 15 to 20 years several methods for producing transgenic plants have been developed, and the present invention also relates to transgenic variants of the claimed apomictic soybean.

Nucleic acids or polynucleotides refer to RNA or DNA that is linear or branched, single or double stranded, or a hybrid thereof. The term also encompasses RNA/DNA hybrids. These terms also encompass untranslated sequences located at both the 3′ and 5′ ends of the coding region of the gene: at least approximately 1000 nucleotides of sequence upstream from the 5′ end of the coding region and at least approximately 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR, and in vitro or in vivo transcription.

One embodiment of the present invention is a process for producing apomictic soybean plants further comprising a desired trait, said process comprising introducing a transgene that confers a desired trait to an apomictic soybean plant of the present invention. Another embodiment is the product produced by this process. In one embodiment the desired trait may be one or more of herbicide resistance, insect resistance, disease resistance, decreased phytate, or modified fatty acid or carbohydrate metabolism. The specific gene may be any known in the art or listed herein, including: a polynucleotide conferring resistance to imidazolinone, dicamba, sulfonylurea, glyphosate, glufosinate, triazine, benzonitrile, cyclohexanedione, phenoxy proprionic acid, and L-phosphinothricin; a polynucleotide encoding a Bacillus thuringiensis polypeptide; a polynucleotide encoding phytase, FAD-2, FAD-3, galactinol synthase, or a raffinose synthetic enzyme; or a polynucleotide conferring resistance to soybean cyst nematode, brown stem rot, Phytophthora root rot, soybean mosaic virus, or sudden death syndrome.

Numerous methods for plant transformation have been developed, including biological and physical plant transformation protocols. See, for example, Miki et al., “Procedures for Introducing Foreign DNA into Plants,” in Methods in Plant Molecular Biology and Biotechnology, Glick and Thompson Eds., CRC Press, Inc., Boca Raton, pp. 67-88 (1993), and Armstrong, “The First Decade of Maize Transformation: A Review and Future Perspective,” Maydica, 44:101-109 (1999). In addition, expression vectors and in vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

A genetic trait which has been engineered into the genome of a particular soybean plant may then be moved into the genome of another variety using traditional breeding techniques that are well known in the plant breeding arts. For example, a backcrossing approach is commonly used to move a transgene from a transformed soybean variety into an already developed soybean variety, and the resulting backcross conversion plant would then comprise the transgene(s).

Various genetic elements can be introduced into the plant genome using transformation. These elements include, but are not limited to, genes, coding sequences, inducible, constitutive and tissue specific promoters, enhancing sequences, and signal and targeting sequences. For example, see the traits, genes, and transformation methods listed in U.S. Pat. No. 6,118,055.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid and can be used alone or in combination with other plasmids to provide transformed soybean plants using transformation methods as described below to incorporate transgenes into the genetic material of the soybean plant(s).

Likewise, by means of the present invention, agronomic genes can be expressed in transformed plants. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with one or more cloned resistance genes to engineer plants that are resistant to specific pathogen strains. See, for example, Jones, et al., Science, 266:789 (1994) (cloning of the tomato Cf-9 gene for resistance to Cladosporium fulvum); Martin, et al., Science, 262:1432 (1993) (tomato Pto gene for resistance to Pseudomonas syringae pv. tomato encodes a protein kinase); Mindrinos, et al., Cell, 78:1089 (1994) (Arabidopsis RSP2 gene for resistance to Pseudomonas syringae); McDowell & Woffenden, Trends Biotechnol., 21(4):178-83 (2003); and Toyoda, et al., Transgenic Res., 11 (6):567-82 (2002).

B. A gene conferring resistance to a pest, such as soybean cyst nematode. See, e.g., PCT Application WO 96/30517 and PCT Application WO 93/19181.

C. A Bacillus thuringiensis protein, a derivative thereof or a synthetic polypeptide modeled thereon. See, for example, Geiser, et al., Gene, 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995, and 31998.

D. A lectin. See, for example, Van Damme, et al., Plant Molec. Biol., 24:25 (1994), who disclose the nucleotide sequences of several Clivia miniata mannose-binding lectin genes.

E. A vitamin-binding protein such as avidin. See, PCT Application US 93/06487, which teaches the use of avidin and avidin homologues as larvicides against insect pests.

F. An enzyme inhibitor, for example, a protease or proteinase inhibitor or an amylase inhibitor. See, for example, Abe, et al., J. Biol. Chem., 262:16793 (1987) (nucleotide sequence of rice cysteine proteinase inhibitor); Huub, et al., Plant Molec. Biol., 21:985 (1993) (nucleotide sequence of cDNA encoding tobacco proteinase inhibitor I); Sumitani, et al., Biosci. Biotech. Biochem., 57:1243 (1993) (nucleotide sequence of Streptomyces nitrosporeus α-amylase inhibitor); and U.S. Pat. No. 5,494,813 (Hepher and Atkinson, issued Feb. 27, 1996).

G. An insect-specific hormone or pheromone, such as an ecdysteroid or juvenile hormone, a variant thereof, a mimetic based thereon, or an antagonist or agonist thereof. See, for example, the disclosure by Hammock, et al., Nature, 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem., 269:9 (1994) (expression cloning yields DNA coding for insect diuretic hormone receptor); Pratt, et al., Biochem. Biophys. Res. Comm., 163:1243 (1989) (an allostatin is identified in Diploptera puntata); Chattopadhyay, et al., Critical Reviews in Microbiology, 30(1):33-54 (2004); Zjawiony, J Nat Prod, 67(2):300-310 (2004); Carlini & Grossi-de-Sa, Toxicon, 40(11):1515-1539 (2002); Ussuf, et al., Curr Sci., 80(7):847-853 (2001); Vasconcelos & Oliveira, Toxicon, 44(4):385-403 (2004). See also, U.S. Pat. No. 5,266,317 to Tomalski, et al., which discloses genes encoding insect-specific, paralytic neurotoxins.

I. An insect-specific venom produced in nature by a snake, a wasp, etc. For example, see, Pang, et al., Gene, 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative, or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase, and a glucanase, whether natural or synthetic. See, PCT Application WO 93/02197 (Scott, et al.), which discloses the nucleotide sequence of a callase gene. DNA molecules which contain chitinase-encoding sequences can be obtained, for example, from the ATCC under Accession Nos. 39637 and 67152. See also, Kramer, et al., Insect Biochem. Molec. Biol., 23:691 (1993), who teach the nucleotide sequence of a cDNA encoding tobacco hornworm chitinase, and Kawalleck, et al., Plant Molec. Biol., 21:673 (1993), who provide the nucleotide sequence of the parsley ubi4-2 polyubiquitin gene, U.S. Pat. Nos. 7,145,060, 7,087,810, and 6,563,020.

L. A molecule that stimulates signal transduction. For example, see the disclosure by Botella, et al., Plant Molec. Biol., 24:757 (1994), of nucleotide sequences for mung bean calmodulin cDNA clones, and Griess, et al., Plant Physiol., 104:1467 (1994), who provide the nucleotide sequence of a maize calmodulin cDNA clone.

M. A hydrophobic moment peptide. See, PCT Application WO 95/16776 and U.S. Pat. No. 5,580,852, which disclose peptide derivatives of tachyplesin which inhibit fungal plant pathogens, and PCT Application WO 95/18855 and U.S. Pat. No. 5,607,914 which teaches synthetic antimicrobial peptides that confer disease resistance.

N. A membrane permease, a channel former or a channel blocker. For example, see the disclosure of Jaynes, et al., Plant Sci, 89:43 (1993), of heterologous expression of a cecropin-β lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See, Beachy, et al., Ann. Rev. Phytopathol., 28:451 (1990). Coat protein-mediated resistance has been conferred upon transformed plants against alfalfa mosaic virus, cucumber mosaic virus, and tobacco mosaic virus.

P. An insect-specific antibody or an immunotoxin derived therefrom. Thus, an antibody targeted to a critical metabolic function in the insect gut would inactivate an affected enzyme, killing the insect. See, Taylor, et al., Abstract #497, Seventh Int'l Symposium on Molecular Plant-Microbe Interactions (Edinburgh, Scotland 1994) (enzymatic inactivation in transgenic tobacco via production of single-chain antibody fragments).

Q. A virus-specific antibody. See, for example, Tavladoraki, et al., Nature, 366:469 (1993), who show that transgenic plants expressing recombinant antibody genes are protected from virus attack.

R. A developmental-arrestive protein produced in nature by a pathogen or a parasite. Thus, fungal endo-α-1,4-D-polygalacturonases facilitate fungal colonization and plant nutrient release by solubilizing plant cell wall homo-α-1,4-D-galacturonase. See, Lamb, et al., Bio/Technology, 10:1436 (1992). The cloning and characterization of a gene which encodes a bean endopolygalacturonase-inhibiting protein is described by Toubart, et al., Plant J., 2:367 (1992).

S. A developmental-arrestive protein produced in nature by a plant. For example, Logemann, et al., Bio/Technology, 10:305 (1992), have shown that transgenic plants expressing the barley ribosome-inactivating gene have an increased resistance to fungal disease.

T. Genes involved in the Systemic Acquired Resistance (SAR) Response and/or the pathogenesis-related genes. Briggs, S., Current Biology, 5(2) (1995); Pieterse & Van Loon, Curr. Opin. Plant Bio., 7(4):456-64 (2004); and Somssich, Cell, 113(7):815-6 (2003).

U. Antifungal genes. See, Cornelissen and Melchers, Plant Physiol., 101:709-712 (1993); Parijs, et al., Planta, 183:258-264 (1991); and Bushnell, et al., Can. J. of Plant Path., 20(2):137-149 (1998). See also, U.S. Pat. No. 6,875,907.

V. Detoxification genes, such as for fumonisin, beauvericin, moniliformin, and zearalenone and their structurally-related derivatives. See, for example, U.S. Pat. No. 5,792,931.

W. Cystatin and cysteine proteinase inhibitors. See, U.S. Pat. No. 7,205,453.

X. Defensin genes. See, WO 03/000863 and U.S. Pat. No. 6,911,577.

Y. Genes conferring resistance to nematodes, and in particular soybean cyst nematodes. See, e.g., PCT Applications WO 96/30517, WO 93/19181, and WO 03/033651; Urwin, et al., Planta, 204:472-479 (1998); Williamson, Curr Opin Plant Bio., 2(4):327-31 (1999).

Z. Genes that confer resistance to Phytophthora Root Rot, such as the Rps1, Rps1a, Rps1b, Rps1c, Rps1d, Rps1e, Rps1k, Rps2, Rps3a, Rps3b, Rps3c, Rps4, Rps5, Rps6, Rps7, and other Rps genes. See, for example, Shoemaker, et al., Phytophthora Root Rot Resistance Gene Mapping in Soybean, Plant Genome IV Conference, San Diego, Calif. (1995).

AA. Genes that confer resistance to Brown Stem Rot, such as described in U.S. Pat. No. 5,689,035 and incorporated by reference for this purpose.

Any of the above-listed disease or pest resistance genes (A-AA) can be introduced into the claimed soybean cultivar through a variety of means including, but not limited to, transformation and crossing.

2. Genes that Confer Resistance to an Herbicide, for Example:

A. An herbicide that inhibits the growing point or meristem, such as an imidazolinone or a sulfonylurea. Exemplary genes in this category code for mutant ALS and AHAS enzyme as described, for example, by Lee, et al., EMBO J., 7:1241 (1988) and Miki, et al., Theor. Appl. Genet., 80:449 (1990), respectively.

B. Glyphosate (resistance conferred by mutant 5-enolpyruvlshikimate-3-phosphate synthase (EPSPS) and aroA genes, respectively) and other phosphono compounds, such as glufosinate (phosphinothricin acetyl transferase (PAT) and Streptomyces hygroscopicus PAT bar genes), pyridinoxy or phenoxy proprionic acids, and cyclohexanediones (ACCase inhibitor-encoding genes). See, for example, U.S. Pat. No. 4,940,835 to Shah, et al., which discloses the nucleotide sequence of a form of EPSPS which can confer glyphosate resistance. U.S. Pat. No. 5,627,061 to Barry, et al., also describes genes encoding EPSPS enzymes. See also, U.S. Pat. Nos. 6,566,587, 6,338,961, 6,248,876, 6,040,497, 5,804,425, 5,633,435, 5,145,783, 4,971,908, 5,312,910, 5,188,642, 4,940,835, 5,866,775, 6,225,114, 6,130,366, 5,310,667, 4,535,060, 4,769,061, 5,633,448, 5,510,471, RE 36,449, RE 37,287, and 5,491,288; and International Publications EP1173580, WO 01/66704, EP1173581, and EP1173582, which are incorporated herein by reference for this purpose. Glyphosate resistance is also imparted to plants that express a gene that encodes a glyphosate oxido-reductase enzyme, as described more fully in U.S. Pat. Nos. 5,776,760 and 5,463,175, which are incorporated herein by reference for this purpose. In addition, glyphosate resistance can be imparted to plants by the over expression of genes encoding glyphosate N-acetyltransferase. See, for example, U.S. Pat. No. 7,462,481. A DNA molecule encoding a mutant aroA gene can be obtained under ATCC Accession No. 39256, and the nucleotide sequence of the mutant gene is disclosed in U.S. Pat. No. 4,769,061 to Comai. European Patent Appl. No. 0 333 033 to Kumada, et al. and U.S. Pat. No. 4,975,374 to Goodman, et al., disclose nucleotide sequences of glutamine synthetase genes which confer resistance to herbicides such as L-phosphinothricin. The nucleotide sequence of a PAT gene is provided in European Patent Appl. No. 0 242 246 to Leemans, et al. DeGreef, et al., Bio/Technology, 7:61 (1989) describe the production of transgenic plants that express chimeric bar genes coding for phosphinothricin acetyl transferase activity. Exemplary of genes conferring resistance to phenoxy proprionic acids and cyclohexones, such as sethoxydim and haloxyfop are the Acc1-S1, Acc1-S2, and Acc2-S3 genes described by Marshall, et al., Theor. Appl. Genet., 83:435 (1992).

C. An herbicide that inhibits photosynthesis, such as a triazine (psbA and gs+ genes) and a benzonitrile (nitrilase gene). Przibila, et al., Plant Cell, 3:169 (1991), describe the transformation of Chlamydomonas with plasmids encoding mutant psbA genes. Nucleotide sequences for nitrilase genes are disclosed in U.S. Pat. No. 4,810,648 to Stalker and DNA molecules containing these genes are available under ATCC Accession Nos. 53435, 67441, and 67442. Cloning and expression of DNA coding for a glutathione S-transferase is described by Hayes, et al., Biochem. J., 285:173 (1992).

D. Acetohydroxy acid synthase, which has been found to make plants that express this enzyme resistant to multiple types of herbicides, has been introduced into a variety of plants. See, Hattori, et al., Mol. Gen. Genet., 246:419 (1995). Other genes that confer tolerance to herbicides include a gene encoding a chimeric protein of rat cytochrome P4507A1 and yeast NADPH-cytochrome P450 oxidoreductase (Shiota, et al., Plant Physiol., 106:17 (1994)); genes for glutathione reductase and superoxide dismutase (Aono, et al., Plant Cell Physiol., 36:1687 (1995)); and genes for various phosphotransferases (Datta, et al., Plant Mol. Biol., 20:619 (1992)).

E. Protoporphyrinogen oxidase (protox) is necessary for the production of chlorophyll, which is necessary for all plant survival. The protox enzyme serves as the target for a variety of herbicidal compounds. These herbicides also inhibit growth of all the different species of plants present, causing their total destruction. The development of plants containing altered protox activity which are resistant to these herbicides are described in U.S. Pat. Nos. 6,288,306, 6,282,837, 5,767,373, and International Publication WO 01/12825.

Any of the above listed herbicide genes (A-E) can be introduced into the apomictic soybean plant of the present invention through a variety of means including but not limited to transformation and crossing.

3. Genes that Confer or Contribute to a Value-Added Trait, such as:

A. Modified fatty acid metabolism, for example, by transforming a plant with an antisense gene of stearyl-ACP desaturase to increase stearic acid content of the plant. See, Knultzon, et al., Proc. Natl. Acad. Sci. USA, 89:2625 (1992).

B. Decreased phytate content: 1) Introduction of a phytase-encoding gene enhances breakdown of phytate, adding more free phosphate to the transformed plant. For example, see, Van Hartingsveldt, et al., Gene, 127:87 (1993), for a disclosure of the nucleotide sequence of an Aspergillus niger phytase gene. 2) Up-regulation of a gene that reduces phytate content. In maize, this, for example, could be accomplished by cloning and then re-introducing DNA associated with one or more of the alleles, such as the LPA alleles, identified in maize mutants characterized by low levels of phytic acid, such as in Raboy, et al., Maydica, 35:383 (1990), and/or by altering inositol kinase activity as in WO 02/059324, U.S. Publ. No. 2003/000901, WO 03/027243, U.S. Publ. No. 2003/0079247, WO 99/05298, U.S. Pat. No. 6,197,561, U.S. Pat. No. 6,291,224, U.S. Pat. No. 6,391,348, WO 2002/059324, U.S. Publ. No. 2003/0079247, WO 98/45448, W O99/55882, and WO 01/04147.

C. Modified carbohydrate composition effected, for example, by transforming plants with a gene coding for an enzyme that alters the branching pattern of starch, or a gene altering thioredoxin, such as NTR and/or TRX (see, U.S. Pat. No. 6,531,648, which is incorporated by reference for this purpose), and/or a gamma zein knock out or mutant, such as cs27 or TUSC27 or en27 (see, U.S. Pat. No. 6,858,778, and U.S. Publ. Nos. 2005/0160488 and 2005/0204418, which are incorporated by reference for this purpose). See, Shiroza, et al., J. Bacteriol., 170:810 (1988) (nucleotide sequence of Streptococcus mutans fructosyltransferase gene); Steinmetz, et al., Mol. Gen. Genet., 200:220 (1985) (nucleotide sequence of Bacillus subtilis levansucrase gene); Pen, et al., Bio/Technology, 10:292 (1992) (production of transgenic plants that express Bacillus licheniformis alpha-amylase); Elliot, et al., Plant Molec. Biol., 21:515 (1993) (nucleotide sequences of tomato invertase genes); Sogaard, et al., J. Biol. Chem., 268:22480 (1993) (site-directed mutagenesis of barley alpha-amylase gene); Fisher, et al., Plant Physiol., 102:1045 (1993) (maize endosperm starch branching enzyme II); WO 99/10498 (improved digestibility and/or starch extraction through modification of UDP-D-xylose 4-epimerase, Fragile 1 and 2, Ref 1, HCHL, C4H); U.S. Pat. No. 6,232,529 (method of producing high oil seed by modification of starch levels (AGP)). The fatty acid modification genes mentioned above may also be used to affect starch content and/or composition through the interrelationship of the starch and oil pathways.

D. Elevated oleic acid via FAD-2 gene modification and/or decreased linolenic acid via FAD-3 gene modification. See, U.S. Pat. Nos. 6,063,947, 6,323,392, and International Publication WO 93/11245. Linolenic acid is one of the five most abundant fatty acids in soybean seeds. The low oxidative stability of linolenic acid is one reason that soybean oil undergoes partial hydrogenation. When partially hydrogenated, all unsaturated fatty acids form trans fats. Soybeans are the largest source of edible-oils in the U.S. and 40% of soybean oil production is partially hydrogenated. The consumption of trans fats increases the risk of heart disease. Regulations banning trans fats have encouraged the development of low linolenic soybeans. Soybeans containing low linolenic acid percentages create a more stable oil requiring hydrogenation less often. This provides trans fat free alternatives in products such as cooking oil.

E. Altering conjugated linolenic or linoleic acid content, such as in WO 01/12800. Altering LEC1, AGP, Dek1, Supera11, mi1ps, and various Ipa genes, such as Ipa1, Ipa3, hpt, or hggt. See, for example, WO 02/42424, WO 98/22604, WO 03/011015, WO 02/057439, WO 03/011015, U.S. Pat. Nos. 6,423,886, 6,197,561, 6,825,397, 7,157,621, U.S. Publ. No. 2003/0079247, and Rivera-Madrid, R., et al., Proc. Natl. Acad. Sci., 92:5620-5624 (1995).

F. Altered antioxidant content or composition, such as alteration of tocopherol or tocotrienols. See, for example, U.S. Pat. Nos. 6,787,683, 7,154,029, WO 00/68393 (involving the manipulation of antioxidant levels through alteration of a phytl prenyl transferase (ppt)); WO 03/082899 (through alteration of a homogentisate geranyl geranyl transferase (hggt)).

G. Altered essential seed amino acids. See, for example, U.S. Pat. No. 6,127,600 (method of increasing accumulation of essential amino acids in seeds); U.S. Pat. No. 6,080,913 (binary methods of increasing accumulation of essential amino acids in seeds); U.S. Pat. No. 5,990,389 (high lysine); U.S. Pat. No. 5,850,016 (alteration of amino acid compositions in seeds); U.S. Pat. No. 5,885,802 (high methionine); U.S. Pat. No. 5,885,801 (high threonine); U.S. Pat. No. 6,664,445 (plant amino acid biosynthetic enzymes); U.S. Pat. No. 6,459,019 (increased lysine and threonine); U.S. Pat. No. 6,441,274 (plant tryptophan synthase beta subunit); U.S. Pat. No. 6,346,403 (methionine metabolic enzymes); U.S. Pat. No. 5,939,599 (high sulfur); U.S. Pat. No. 5,912,414 (increased methionine); U.S. Pat. No. 5,633,436 (increasing sulfur amino acid content); U.S. Pat. No. 5,559,223 (synthetic storage proteins with defined structure containing programmable levels of essential amino acids for improvement of the nutritional value of plants); U.S. Pat. No. 6,194,638 (hemicellulose); U.S. Pat. No. 7,098,381 (UDPGdH); U.S. Pat. No. 6,194,638 (RGP); U.S. Pat. Nos. 6,399,859, 6,930,225, 7,179,955, and 6,803,498; U.S. Publ. No. 2004/0068767; WO 99/40209 (alteration of amino acid compositions in seeds); WO 99/29882 (methods for altering amino acid content of proteins); WO 98/20133 (proteins with enhanced levels of essential amino acids); WO 98/56935 (plant amino acid biosynthetic enzymes); WO 98/45458 (engineered seed protein having higher percentage of essential amino acids); WO 98/42831 (increased lysine); WO 96/01905 (increased threonine); WO 95/15392 (increased lysine); WO 01/79516; and WO 00/09706 (Ces A: cellulose synthase).

### Methods for Soybean Transformation

Numerous methods for plant transformation have been developed including biological and physical plant transformation protocols. See, for example, Miki, et al., “Procedures for Introducing Foreign DNA into Plants,” in Methods in Plant Molecular Biology and Biotechnology, Glick and Thompson Eds., CRC Press, Inc., Boca Raton, pp. 67-88 (1993). In addition, expression vectors and in-vitro culture methods for plant cell or tissue transformation and regeneration of plants are available. See, for example, Gruber, et al., “Vectors for Plant Transformation,” in Methods in Plant Molecular Biology and Biotechnology, Glick and Thompson Eds., CRC Press, Inc., Boca Raton, pp. 89-119 (1993).

A. Agrobacterium-mediated Transformation—One method for introducing an expression vector into plants is based on the natural transformation system of Agrobacterium. See, for example, Horsch, et al., Science, 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci., 10:1 (1991). Descriptions of Agrobacterium vector systems and methods for Agrobacterium-mediated gene transfer are provided by Gruber, et al., supra, Miki, et al., supra, and Moloney, et al., Plant Cell Reports, 8:238 (1989). See also, U.S. Pat. No. 5,563,055 (Townsend and Thomas), issued Oct. 8, 1996.

B. Direct Gene Transfer—Several methods of plant transformation, collectively referred to as direct gene transfer, have been developed as an alternative to Agrobacterium-mediated transformation. A generally applicable method of plant transformation is microprojectile-mediated transformation where DNA is carried on the surface of microprojectiles measuring 1 to 4 μm. The expression vector is introduced into plant tissues with a biolistic device that accelerates the microprojectiles to speeds of 300 to 600 m/s which is sufficient to penetrate plant cell walls and membranes. Sanford, et al., Part. Sci. Technol., 5:27 (1987); Sanford, J. C., Trends Biotech., 6:299 (1988); Klein, et al., Bio/Tech., 6:559-563 (1988); Sanford, J. C., Physiol Plant, 7:206 (1990); Klein, et al., Biotechnology, 10:268 (1992). See also, U.S. Pat. No. 5,015,580 (Christou, et al.), issued May 14, 1991 and U.S. Pat. No. 5,322,783 (Tomes, et al.), issued Jun. 21, 1994.

Another method for physical delivery of DNA to plants is sonication of target cells. Zhang, et al., Bio/Technology, 9:996 (1991). Alternatively, liposome and spheroplast fusion have been used to introduce expression vectors into plants. Deshayes, et al., EMBO J., 4:2731 (1985); Christou, et al., Proc Natl. Acad. Sci. USA, 84:3962 (1987). Direct uptake of DNA into protoplasts using CaCl₂ precipitation, polyvinyl alcohol or poly-L-ornithine have also been reported. Hain, et al., Mol. Gen. Genet., 199:161 (1985) and Draper, et al., Plant Cell Physiol., 23:451 (1982). Electroporation of protoplasts and whole cells and tissues have also been described (Donn, et al., In Abstracts of VIIth International Congress on Plant Cell and Tissue Culture IAPTC, A2-38, p. 53 (1990); D'Halluin, et al., Plant Cell, 4:1495-1505 (1992); and Spencer, et al., Plant Mol. Biol., 24:51-61 (1994)).

Following transformation of soybean target tissues, expression of the above-described selectable marker genes allows for preferential selection of transformed cells, tissues, and/or plants, using regeneration and selection methods well known in the art.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular soybean line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple x by y cross or the process of backcrossing depending on the context.

Genetic Marker Profile through SSR and First Generation Progeny

In addition to phenotypic observations, a plant can also be identified by its genotype. The genotype of a plant can be characterized through a genetic marker profile which can identify plants of the same variety, or a related variety, or be used to determine or validate a pedigree. Genetic marker profiles can be obtained by techniques such as Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and Single Nucleotide Polymorphisms (SNPs). For example, see, Cregan, et al., “An Integrated Genetic Linkage Map of the Soybean Genome,” Crop Science, 39:1464-1490 (1999) and Berry, et al., “Assessing Probability of Ancestry Using Simple Sequence Repeat Profiles: Applications to Maize Inbred Lines and Soybean Varieties,” Genetics, 165:331-342 (2003), each of which are incorporated by reference herein in their entirety.

Particular markers used for these purposes are not limited to any particular set of markers, but are envisioned to include any type of marker and marker profile which provides a means of distinguishing varieties.

### EXAMPLES

The data set forth in the Examples demonstrate the outstanding traits and characteristics of selections of apomictic soybean plants 7B1 and S-201, and apomictic soybean plants obtained through crossing of apomictic lines 7B1 or S-201 as the male or female parent with a number of different female or male soybean lines, or by selfing. Crosses and evaluations were performed in Brookston, Ind. in 2007-2011. Plants were crossed and seed was allowed set on the female plant, and the seed was then grown and the resulting plants were observed and selected; observations of these crosses and/or selections were used to develop the tables below. As shown in the Examples and accompanying Tables, the apomictic soybean plants produced demonstrate very high pod count and hybrid vigor.

### Example 1

### Apomictic Soybean Produced by Cross of 93×17×7B1

The following example describes the traits of an apomictic soybean plant produced by crossing male apomictic line 7B1 with female soybean line 93×17. As shown in Table 1, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 1

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x17 X 7B1 | 24 | 14 | 131 | 9.3 | 65 | 419 | 6.5 |

### Example 2

### Single Plant Selection of Apomictic Soybean Line 7B1

The following example describes the traits of a single plant selection of the apomictic soybean plant 7B1 designated 7B1 tag 7-2, which was a single plant selection from the 7^{th} plant used in an emasculation study and came from the second pod produced. As shown in Table 2, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 2

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| 7B1 tag 7-2 | 32 | 17 | 138 | 8.1 | 53 | 290 | 5.5 |

### Example 3

### Apomictic Soybean Produced by Cross of 93×12×7B1

The following example describes the traits of the apomictic soybean plant produced by crossing male apomictic line 7B1 with female soybean line 93×12. As shown in Table 3, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 3

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x12 X 7B1 | 23 | 16 | 81 | 5.1 | 42 | 245 | 5.8 |

### Example 4

### Single Plant Selection of Apomictic Soybean Line S-201

The following example describes the traits of a single plant selection of S-201 designated SS 40. As shown in Table 4, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 4

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| SS 40 | 33 | 13 | 92 | 7.1 | 66 | 342 | 5.2 |

### Example 5

### Single Plant Selection of Apomictic Soybean Line S-201

The following example describes the traits of a single plant selection of S-201 designated DOS85, which was the 85^{th} selection of a double ovule seed in 2009. As shown in Table 5, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 5

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| DOS85 | 26 | 18 | 157 | 8.7 | 50 | 345 | 6.9 |

### Example 6

### Self Test of Apomictic Soybean Line 7B1

The following example describes the traits of apomictic soybean plant 7B1 designated 7B1-3, which was used in a self test for an emasculation study. As shown in Table 6, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 6

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| 7B1-3 | 34 | 18 | 126 | 7.0 | 82 | 438 | 5.3 |

### Example 7

### Self Test of Apomictic Soybean Line 7B1

The following example describes the traits of the apomictic soybean plant 7B1 designated 7B1 tag 7-1, which was used in a self test for an emasculation study. As shown in Table 7, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 7

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| 7B1 tag 7-1 | 21 | 16 | 190 | 11.9 | 54 | 433 | 8 |

### Example 8

### Apomictic Soybean Produced by Cross of 93×25×7B1

The following example describes the traits of the apomictic soybean plant produced by crossing male apomictic line 7B1 with female soybean line 93×25. As shown in Table 8, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 8

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x25 X 7B1 | 26 | 14 | 93 | 6.6 | 88 | 331 | 3.8 |

### Example 9

### Apomictic Soybean Produced by Cross of 93×58×7B1

The following example describes the traits of the apomictic soybean plant produced by crossing male apomictic line 7B1 with female soybean line 93×58. As shown in Table 9, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 9

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x58 X 7B1 | 27 | 16 | 84 | 5.3 | 64 | 251 | 3.9 |

### Example 10

### Apomictic Soybean Produced by Cross of 93×3×7B1

The following example describes the traits of the apomictic soybean plant produced by crossing male apomictic line 7B1 with female soybean line 93×3. As shown in Table 10, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 10

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x3 X 7B1 | 22 | 17 | 93 | 5.5 | 83 | 366 | 4.4 |

### Example 11

### Apomictic Soybean Produced by Cross of 93×44×7B1

The following example describes the traits of the apomictic soybean plant produced by crossing male apomictic line 7B1 with female soybean line 93×44. As shown in Table 11, the breeding cross used is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 11

| | Plant | | | Avg. MS | | | Avg. |
|---|---|---|---|---|---|---|---|
| | height | MS | MS | pods/ | Total | Total | Pods/ |
| Breeding cross | (in.) | nodes | pods | node | nodes | pods | node |
| 93x44 X 7B1 | 28 | 17 | 160 | 9.4 | 93 | 479 | 5.2 |

### Example 12

### Single Plant Selection of Apomictic Soybean Line S-201

The following example describes the traits of a single plant selection of S-201 designated SS 40-2, which is the same line presented in Example 4, but grown one year later. As shown in Table 12, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 12

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| SS 40-2 | 34 | 21 | 191 | 9.1 | 58 | 463 | 8 |

### Example 13

### Apomictic Soybean Line S-201

The following example describes the traits of the apomictic soybean line S-201. As shown in Table 13, the plant is listed in column 1, the plant height in inches is listed in column 2, the number of nodes on the main stem (MS) is listed in column 3, the number of pods on the main stem (MS) is listed in column 4, the average number of pods per node on the main stem (MS) is listed in column 5, the total number of nodes per plant is listed in column 6, the total number of pods per plant is listed in column 7, and the average number of total pods per total nodes is listed in column 8.

TABLE 13

| | Plant | | | Avg. MS | | | Avg. |
|---|---|---|---|---|---|---|---|
| | height | MS | MS | pods/ | Total | Total | Pods/ |
| Plant | (in.) | nodes | pods | node | nodes | pods | node |
| S-201 | 25 | 19 | 155 | 8.2 | 55 | 320 | 5.8 |

### Example 14

### Apomictic Soybean Produced by Cross of KK17×7B1

The following example describes the traits of the apomictic soybean plant produced by crossing male apomictic line 7B1 with female soybean line KK17. As shown in Table 14, the breeding cross used is listed in column 1, the plant height in inches is listed in column 2, the total number of nodes per plant is listed in column 3, the total number of pods per plant is listed in column 4, and the average number of total pods per total nodes is listed in column 5.

TABLE 14

| | Plant | Total | Total | Avg. Pods/ |
|---|---|---|---|---|
| Breeding cross | height (in.) | nodes | pods | node |
| KK17 X 7B1 | 44 | 25 | 326 | 13 |

### Example 15

### Apomictic Soybean Produced by Cross of 01×437×7B1

The following example describes the traits of two apomictic soybean plants produced by crossing male apomictic line 7B1 with female soybean line 01×437. As shown in Table 15, the breeding cross used is listed in column 1, the plant height in inches is listed in column 2, the total number of nodes per plant is listed in column 3, the total number of pods per plant is listed in column 4, and the average number of total pods per total nodes is listed in column 5.

TABLE 15

| | Plant | Total | Total | Avg. Pods/ |
|---|---|---|---|---|
| Breeding cross | height (in.) | nodes | pods | node |
| 01x437 X 7B1 | 26 | 25 | 520 | 20.1 |
| 01x437 X 7B1 | 24 | 21 | 419 | 19.9 |

### Example 16

### Apomictic Soybean Produced by Cross of T3001×7B1

The following example describes the traits of the apomictic soybean plant produced by crossing male apomictic line 7B1 with female soybean line T3001. As shown in Table 16, the breeding cross used is listed in column 1, the plant height in inches is listed in column 2, the total number of nodes per plant is listed in column 3, the total number of pods per plant is listed in column 4, and the average number of total pods per total nodes is listed in column 5.

TABLE 16

| | Plant | Total | Total | Avg. Pods/ |
|---|---|---|---|---|
| Breeding cross | height (in.) | nodes | pods | node |
| T3001 X 7B1 | 22 | 25 | 366 | 14.6 |

As shown in the preceding Tables 1-16, the apomictic soybean varieties of the present invention show a greatly increased number of pods per plant and pods per node.

### Example 17

### Capturing Hybrid Vigor through Crossbreeding with Apomictic Lines

Example 17 describes capturing hybrid vigor by crossing a male commercial variety, Dwight, with apomictic line 7B1 as the female parent. Hybrid vigor refers to the improved performance in one or more traits and is expressed by specific F₁ hybrids, resulting from their genetic heterogeneity. This improved performance is typically reduced or eliminated when planting the progeny seed of a hybrid. In the present invention, an apomictic F₁ improved performance is maintained in the following season when the harvested F₁ apomictic seeds are planted. In this example, apomictic line 7B1 was first used as a male parent in a cross with proprietary female line Fem84C; an apomictic plant from this cross was used as a female parent in the cross with Dwight. In this Example and Table, the commercial variety Dwight has an estimated average pod count of 50 pods per plant. As shown in Table 17, the breeding cross used is listed in column 1, the plant height in inches is listed in column 2, the total number of nodes per plant is listed in column 3, the total number of pods per plant is listed in column 4, and the average number of total pods per total nodes is listed in column 5.

TABLE 17

| | Plant | Total | Total | Avg. Pods/ |
|---|---|---|---|---|
| Breeding cross | height (in.) | nodes | pods | node |
| Dwight | — | — | 50 | — |
| (Fem84C x 7B1) X Dwight | 29 | 25 | 422 | 16.9 |

As shown in Example 17, any hybrid vigor was captured through crossbreeding with apomictic line 7B1, as shown by the unexpectedly high total number of pods per plant. In summary, Examples 1, 3, 8-11 and 14-17 all show examples of F₁ apomictic lines that capture and maintain any F₁ hybrid vigor over 2 or more subsequent planting generations. The apomictic F₁ seed that is planted will produce plants with seed that when harvested and grown will have the same genotype as the F₁ parent line, so hybrid vigor is maintained in the harvested seed over multiple planting generations.

### Example 18

### Emasculation of Apomictic Line 7B1

The following example describes data obtained from emasculation of apomictic line 7B1. In this example, nine plants of apomictic line 7B1 were grown and all of the pollen was removed from a number of flowers on each of the plants and tags indicated where each emasculated flower was located. The plants were then observed for pod set at the site of the emasculated flowers indicated by the tags. It should be noted here that emasculation destroys the male portion of the flower; a flower without male parts should not be compared with other flowers that have these parts. As shown in Table 18, the plant is listed in column 1, the number of flowers that were emasculated is listed in column 2, the pods that set from the emasculated flowers is listed in column 3, and the percent pod set is listed in column 4.

TABLE 18

| | No. of flowers | Pods from | % pod |
|---|---|---|---|
| Plant | emasculated | flowers | set |
| 1 | 12 | 5 | 42 |
| 2 | 23 | 11 | 48 |
| 3 | 10 | 4 | 40 |
| 4 | 23 | 5 | 22 |
| 5 | 3 | 2 | 67 |
| 7 | 12 | 8 | 67 |
| 8 | 7 | 3 | 43 |
| 9 | 15 | 2 | 13 |
| 10 | 17 | 4 | 24 |
| Total average | 122 | 43 | 33 |

As shown in Example 18, apomictic soybean line 7B1 has a high percent pod set for flowers that were emasculated.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### Deposit Information

A deposit of the G. Robert Taylor, acting as KenAvis Corporation, LLC, proprietary S-201 soybean seed containing the mutant allele AP1 of the present invention disclosed above and recited in the appended claims has been made with the American Type Culture Collection (ATCC), 10801 University Boulevard, Manassas, Va. 20110. The date of deposit was May 24, 2011. The deposit of 2,500 seeds was taken from the same deposit maintained by inventor G. Robert Taylor, acting as KenAvis Corporation, LLC since prior to the filing date of U.S. patent application Ser. No. 13/102,591. All restrictions will be irrevocably removed upon granting of a patent, and the deposit is intended to meet all of the requirements of 37 C.F.R. §§1.801-1.809. The ATCC Accession Number is PTA-11892. The deposit will be maintained in the depository for a period of thirty years, or five years after the last request, or for the enforceable life of the patent, whichever is longer, and will be replaced as necessary during that period.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

1. A method of producing a soybean apomictic plant that exhibits a greater percent conversion and a greater number of pods per plant than commercial soybean varieties comprising the steps of;
a. selecting a plurality of double ovule seeds;
b. growing said seeds into soybean plants; and
c. selecting soybean plants having a percent conversion greater than about 51% and at least 60 pods per plant, wherein a soybean apomictic plant is produced.

2. The method of claim 1, wherein plants are selected having a percent conversion greater than about 60%.

3. The method of claim 2, wherein plants are selected having at least 100 pods per plant.

4. The method of claim 1 further comprising the steps of:
a. cross-breeding the selected apomictic soybean plant as a male with a different soybean plant as the female;
b. harvesting seeds from the female soybean plant;
c. growing said seeds from the female soybean plant; and
d. selecting soybean plants having a percent conversion greater than approximately 51% and at least 60 pods per plant.

5. The method of claim 4, wherein plants are selected having a percent conversion greater than about 60%.

6. The method of claim 5, wherein plants are selected having at least 100 pods per plant.

7. The method of claim 1 further comprising the steps of:
a. cross-breeding the selected apomictic soybean plant as a female with a different soybean plant as the male;
b. harvesting seeds from the female soybean plant;
c. growing said seeds from the female soybean plant; and
d. selecting soybean plants having a percent conversion greater than approximately 51% and at least 60 pods per plant.

8. The method of claim 7, wherein plants are selected having a percent conversion greater than about 60%.

9. The method of claim 8, wherein plants are selected having at least 100 pods per plant.

10. The method of claim 1, wherein the apomictic soybean plants have a determinate growth habit and are adapted to maturity groups V through X.

11. The method of claim 1, wherein the apomictic soybean plants have a percent conversion of between about 51% and about 70%.

12. The method of claim 1, wherein the apomictic soybean plants have a percent conversion of between about 70% and about 90%.

13. The method of claim 1 wherein the apomictic soybean plants have a percent conversion of between about 90% and about 100%.

14. The method of claim 1, wherein the apomictic soybean plants have between about 60 and about 100 pods per plant.

15. The method of claim 1, wherein the apomictic soybean plants have between about 100 and about 200 pods per plant.

16. The method of claim 1, wherein the apomictic soybean plants have between about 200 and about 300 pods per plant.

17. The method of claim 1, wherein the apomictic soybean plants have between about 300 and about 400 pods per plant.

18. The method of claim 1, wherein the apomictic soybean plants have greater than 400 pods per plant.

19. The method of claim 1, wherein in the apomictic soybean plant is of variety S-201 deposited under ATCC Accession No. PTA-11892.

20. An apomictic soybean plant produced by the method of claim 1.

21. An apomictic soybean seed, wherein the seed is produced by the apomictic soybean plant of claim 20.

22. An apomictic soybean plant of soybean variety S-201, representative seed of said soybean variety having been deposited under ATCC Accession No. PTA-11892.

23. An apomictic soybean plant, wherein the apomictic soybean plant is a progeny of the soybean variety S-201 of claim 22.

24. A seed of soybean variety S-201, representative seed of said soybean variety having been deposited under ATCC Accession No. PTA-11892.

25. A method of producing soybean seed, said method comprising crossing the apomictic soybean variety of claim 22 with itself or a second soybean plant and harvesting the resultant seed.

26. A method of producing hybrid soybean seed, said method comprising crossing the plant of claim 22 with a second, distinct soybean plant and harvesting the resultant F₁ hybrid seed.

27. An F₁ hybrid seed produced by the method of claim 26.

28. An apomictic soybean plant, or part thereof, produced by growing said seed of claim 27.

29. A method of introducing a desired trait into the apomictic soybean plant of claim 22, said method comprising introducing a transgene conferring the desired trait, wherein the desired trait is selected from the group consisting of male sterility, herbicide tolerance, insect or pest resistance, disease resistance, modified fatty acid metabolism, modified carbohydrate metabolism and modified soybean fiber characteristics.

30. The method of claim 29, wherein the desired trait is herbicide tolerance and the tolerance is conferred to an herbicide selected from the group consisting of glyphosate, sulfonylurea, imidazalinone, dicamba, glufosinate, phosphinothricin, phenoxy proprionic acid, cycloshexone, triazine, benzonitrile and broxynil.

31. A plant produced by the method of claim 29, wherein the plant has the desired trait.

32. A method of producing an inbred soybean plant derived from the apomictic soybean plant of claim 22, said method comprising the steps of:
a. producing a progeny plant derived from soybean variety S-201 by crossing a plant of the soybean variety S-201 with a soybean plant of a second variety;
b. crossing said progeny plant with itself or a second plant to produce a seed of a progeny plant of a subsequent generation;
c. growing a progeny plant of a subsequent generation from said seed and crossing the progeny plant of a subsequent generation with itself or a second plant; and
d. repeating the last two steps until an inbred soybean plant derived from the soybean variety S-201 is produced.

33. A method of producing a commodity plant product comprising obtaining the apomictic soybean plant of claim 22, or a part thereof, and producing said commodity plant product therefrom.

34. The method of claim 33, wherein the commodity plant product is protein concentrate, protein isolate, soybean hulls, meal, flour or oil.

35. A soybean plant of a soybean variety having a characteristic of a frequency of double ovule seeds of between about 1 to about 5 double ovule seeds per thousand seeds, wherein said frequency of double ovule seeds is conferred by mutant allele AP1, representative sample of seed containing said mutant allele was deposited under ATCC Accession No. PTA-11892.

36. The soybean plant of claim 35, wherein said frequency of double ovule seeds is between about 1 to about 4 double ovule seeds per thousand seeds.

37. The soybean plant of claim 35, wherein said frequency of double ovule seeds is between about 1 to about 3 double ovule seeds per thousand seeds.

38. The soybean plant of claim 35, wherein said frequency of double ovule seeds is between about 1 to about 2 double ovule seeds per thousand seeds.

39. The soybean plant of claim 35, wherein said frequency of double ovule seeds on average is about 1.3 double ovule seeds per thousand seeds.

40. The soybean plant of claim 35, wherein said plant has the further characteristic of apomixis.

41. The soybean plant of claim 36, wherein said plant has the further characteristic of apomixis.

42. The soybean plant of claim 37, wherein said plant has the further characteristic of apomixis.

43. The soybean plant of claim 38, wherein said plant has the further characteristic of apomixis.

44. The soybean plant of claim 39, wherein said plant has the further characteristic of apomixis.

45. A tissue culture produced from protoplasts or cells from the plant of claim 22, wherein said cells or protoplasts are produced from a plant part selected from the group consisting of leaf, pollen, ovule, embryo, cotyledon, hypocotyl, meristematic cell, root, root tip, pistil, anther, flower, seed, shoot, stem, pod and petiole.

46. A soybean plant regenerated from the tissue culture of claim 45.

47. A soybean seed containing a mutant allele designated AP1, wherein a representative sample of seed containing said allele AP1 was deposited under ATCC Accession No. PTA-11892.

48. A soybean plant, or part thereof, produced by growing the seed of claim 47.

49. A determinate apomictic soybean plant having increased yield when compared to commercial soybean varieties grown in the United States maturity groups V through X.
